(19) United States
(12) Patent Application Publication
(10) Pub. No.: US 2025/0269093 A1
(43) Pub. Date: Aug. 28, 2025

(54) METHODS OF ENHANCING ANTIMICROBIAL EFFICACY OF LOCAL ANESTHETICS

(71) Applicant: Chinook Contract Research Inc., Victoria (CA)

(72) Inventors: Nicholas Allan, Victoria (CA); Joseph Ross, Calgary (CA); Merle Olson, Calgary (CA)

(21) Appl. No.: 19/199,274

(22) Filed: May 5, 2025

Related U.S. Application Data
(63) Continuation of application No. PCT/CA2024/050943, filed on Jul. 15, 2024.
(60) Provisional application No. 63/513,769, filed on Jul. 14, 2023.

(51) Int. Cl. A61L 29/16

(52) U.S. Cl. CPC A61L 29/16; A61L 2300/204; A61L 2300/214; A61L 2300/402; A61L 2300/406; A61L 2300/45

(57) ABSTRACT

A method, use, and a drug-eluting medical device for enhancing antimicrobial efficacy of a local anesthetic. The method comprises providing a drug-eluting medical device containing the local anesthetic within an elastomeric material, and administering the drug-eluting medical device to a subject such that it contacts a tissue and elutes the local anesthetic thereto; wherein the eluted local anesthetic provides an enhanced antimicrobial efficacy as compared to the same local anesthetic when not delivered by the drug-eluting medical device.

### CROSS-REFERENCE TO RELATED APPLICATION

This application claims priority to and benefit from U.S. Patent Application No. 63/513,769 filed on Jul. 14, 2023, which is hereby incorporated by reference in its entirety.

### FIELD OF THE DISCLOSURE

The present disclosure relates generally to methods for enhancing antimicrobial efficacy of local anesthetics, and in particular, to local anesthetics eluted from a drug-eluting medical device.

### BACKGROUND

Topical, implanted, or indwelling medical devices including urinary catheters provide important medical interventions. However, they have the inherent drawbacks of having pain associated with the placement of the device, and presenting a site for opportunistic microbial ingress and subsequent infection.

A medical device or a material technology that incorporates a potent local anesthetic and antimicrobial agent is thus needed to address both of these issues. Furthermore, a single agent that provides both the anesthetic and antimicrobial properties incorporated into a medical device may be a simple and efficient solution to this problem. In particular, use of an anesthetic as an antimicrobial agent providing enhanced antimicrobial efficacy as opposed to an antibiotic may limit the potential spread of antimicrobial resistance to various classes of antibiotics.

Lidocaine is a well-known local anesthetic that works to prevent pain signals from beginning or being transmitted by blocking the sodium channels in the affected area.

Local anesthetics including lidocaine are used in many different areas including ligation. In particular, local anesthetics are used in elastration, which is ligation with an elastic band, a bloodless method of male castration and docking commonly used for livestock. Elastration involves placing a tight elastic band, or elastrator, around the body part of an animal to reduce the blood flow to the part so that it withers and falls off. To reduce the discomfort to the animal during this procedure, either the elastration band or the body part or both can be coated with an anesthetic such as lidocaine prior to placement on the animal.

However, currently available medical devices are not ideal for possessing both pain relief and antimicrobial and/or anti-biofilm properties that can elute from the devices to kill or inhibit growth of a panel of wound-infecting microbial pathogens. In particular, currently available devices do not provide sufficient antimicrobial efficacy of an anesthetic. Devices and methods for enhancing antimicrobial efficacy of anesthetics are thus needed.

### SUMMARY

The present disclosure relates to methods, uses and devices for enhancing antimicrobial efficacy of a local anesthetic using a drug-eluting medical device comprising the local anesthetic.

According to one aspect of this disclosure, there is provided a method for enhancing antimicrobial efficacy of a local anesthetic, the method comprising: providing a drug-eluting medical device containing the local anesthetic, the drug eluting medical device comprising the local anesthetic within an elastomeric material; and administering the drug-eluting medical device to a subject such that it contacts a tissue and elutes the local anesthetic thereto; wherein the eluted local anesthetic provides an enhanced antimicrobial efficacy as compared to the same local anesthetic when not delivered by the drug-eluting medical device.

In an embodiment, the drug-eluting medical device is a ring or a band.

In an embodiment, the drug-eluting medical device is a topical, implanted, or indwelling medical device.

In an embodiment, the drug-eluting medical device is a catheter, such as a urinary catheter. The catheter may, for example, be an internal catheter, an external catheter, or a catheter that is both internal and external. The catheter may, for example, be an indwelling catheter, a condom catheter, or an intermittent self-catheter.

In an embodiment, the local anesthetic is a hydrophobic, a hydrophilic, or a lipophilic local anesthetic.

In an embodiment, the local anesthetic is a base form or hydrochloride form of lidocaine, bupivacaine, proparacaine, benzocaine, mepivacaine, bupivacaine liposomal, ropivacaine, chloroprocaine hydrochloride, levobupivacaine, or any combination thereof. In a particular embodiment, the local anesthetic is the base form of the local anesthetic. In a particular embodiment, the local anesthetic is the hydrochloride form of the local anesthetic.

In an embodiment, the local anesthetic is lidocaine. The lidocaine may, for example, be an acidic form or a free base form. In a preferred embodiment, the lidocaine is free base lidocaine.

In an embodiment, the local anesthetic is bupivacaine. The bupivacaine may, for example, be an acidic form or a free base form. In a preferred embodiment, the bupivacaine is free base lidocaine.

In an embodiment, the tissue is epithelial tissue, such as for example skin, the lining of intestines, the lining of the respiratory tract, the lining of blood vessels, the lining of the abdominal cavity, periodontium, gingiva, or any other epithelial tissue. In a particular embodiment, the epithelial tissue is the outer layer of skin.

In an embodiment, the drug-eluting medical device further contains a tissue permeator within the elastomeric material. The tissue permeator may, for example, be isopropyl myristate, fatty acids, fatty acid esters, poloxamers, triglycerides, n-methyl pyrrolidone, terpineol, limonene, dimethyl sulfoxide, dimethylacetamide, or any combination thereof.

In an embodiment, the elastomeric material is a rubber or polymeric material. The rubber or polymeric material may, for example, be selected from a natural rubber, a synthetic rubber, a silicone, a polybutadiene, a polyisoprene, a polychloroprene, a nitrile, a poly(styrene-butadiene-styrene) (SBS), a styrene-ethylene-butylene-styrene (SEBS), an ethylene-propylene-diene monomer rubber (EPDM), a polyurethane, a woven material, a natural fiber, a synthetic fiber, and any combination thereof. In an embodiment, the rubber or polymeric material is latex rubber.

In an embodiment, the drug-eluting medical device further contains a vasoconstrictor within the elastomeric material. The vasoconstrictor may, for example, be epinephrine, pseudoephedrine, phenylephrine, thromboxane, or angiotensin.

In an embodiment, the drug-eluting medical device further contains an antimicrobial agent within the elastomeric material. The antimicrobial agent may, for example, be an alcohol, a cationic, surface-active quaternary ammonium compound, a bisphenol, a chloride compound, an iodine or iodized compound, an aldehyde, a mercurial, an oligodynamic metal, a heavy metal, an acid, a dye, an antibiotic drug, a chemotherapeutic drug, a coal-tar additive, an aromatic oil, or any other suitable agent or compound having antimicrobial activity. In an embodiment, the antimicrobial agent is high valency silver or silver sulphadiazine.

According another aspect of this disclosure, there is provided a use of a drug-eluting medical device for enhancing the antimicrobial efficacy of a local anesthetic, wherein the drug eluting medical device comprises the local anesthetic within an elastomeric material from which it is eluted and wherein the eluted local anesthetic provides an enhanced antimicrobial efficacy as compared to the same local anesthetic when not delivered by the drug-eluting medical device.

In an embodiment, the drug-eluting medical device is a tube, a ring, or a band.

In an embodiment, the drug-eluting medical device is a topical, implanted, or indwelling medical device.

In an embodiment, the drug-eluting medical device is a catheter, such as a urinary catheter. The catheter may, for example, be an internal catheter, an external catheter, or a catheter that is both internal and external. The catheter may, for example, be an indwelling catheter, a condom catheter, or an intermittent self-catheter.

In an embodiment, the local anesthetic is a hydrophobic, a hydrophilic, or a lipophilic local anesthetic.

In an embodiment, the local anesthetic is a base form or hydrochloride form of lidocaine, bupivacaine, proparacaine, benzocaine, mepivacaine, bupivacaine liposomal, ropivacaine, chloroprocaine hydrochloride, levobupivacaine, or any combination thereof. In a particular embodiment, the local anesthetic is the base form of the local anesthetic. In a particular embodiment, the local anesthetic is the hydrochloride form of the local anesthetic.

In an embodiment, the local anesthetic is lidocaine. The lidocaine may, for example, be an acidic form or a free base form. In a preferred embodiment, the lidocaine is free base lidocaine.

In an embodiment, the local anesthetic is bupivacaine. The bupivacaine may, for example, be an acidic form or a free base form. In a preferred embodiment, the bupivacaine is free base lidocaine.

In an embodiment, the drug-eluting medical device further contains a tissue permeator within the elastomeric material. The tissue permeator may, for example, be isopropyl myristate, fatty acids, fatty acid esters, poloxamers, triglycerides, n-methyl pyrrolidone, terpineol, limonene, dimethyl sulfoxide, dimethylacetamide, or any combinations thereof.

In an embodiment, the elastomeric material is a rubber or polymeric material. The rubber or polymeric material may, for example, be selected from a natural rubber, a synthetic rubber, a silicone, a polybutadiene, a polyisoprene, a polychloroprene, a nitrile, a poly(styrene-butadiene-styrene) (SBS), a styrene-ethylene-butylene-styrene (SEBS), an ethylene-propylene-diene monomer rubber (EPDM), a polyurethane, a woven material, a natural fiber, a synthetic fiber, and any combinations thereof. In an embodiment, the rubber or polymeric material is latex rubber.

In an embodiment, the drug-eluting medical device further comprises a vasoconstrictor within the elastomeric material. The vasoconstrictor may, for example, be epinephrine, pseudoephedrine, phenylephrine, thromboxane, or angiotensin.

In an embodiment, the drug-eluting medical device further contains an antimicrobial agent within the elastomeric material. The antimicrobial agent may, for example, be an alcohol, a cationic, surface-active quaternary ammonium compound, a bisphenol, a chloride compound, an iodine or iodized compound, an aldehyde, a mercurial, an oligodynamic metal, a heavy metal, an acid, a dye, an antibiotic drug, a chemotherapeutic drug, a coal-tar additive, an aromatic oil, or any other suitable agent or compound having antimicrobial activity. In an embodiment, the antimicrobial agent is high valency silver or silver sulphadiazine.

According to another aspect of this disclosure, there is provided a drug-eluting medical device providing both anesthetic and antimicrobial effects, the drug-eluting medical device comprising a local anesthetic contained within an elastomeric material from which it is eluted.

Other aspects and embodiments of the disclosure are evident in view of the detailed description provided herein.

### BRIEF DESCRIPTION OF THE DRAWINGS

These and other features of the present disclosure will become more apparent in the following detailed description in which reference is made to the appended drawings. The appended drawings illustrate one or more embodiments of the present disclosure by way of example only and are not to be construed as limiting the scope of the present disclosure.

FIG. 1 is a photo of a zone of inhibition (ZOI) schematic for E. coli, American Type Culture Collection (ATCC) 25922, showing ZOI of a lidocaine loaded band (LLB) having impregnated therein free-base lidocaine; a no lidocaine “mock processed” control (CON); an unprocessed off-the-shelf control (OTS); and an aqueous eluate from a LLB (EL).

FIG. 2 is a photo showing soaked bands.

FIG. 3 is a photo showing a comparison of a control band and a lidocaine loaded band.

FIG. 4 is a flow diagram representing an experimental process for a ZOI antimicrobial assay.

FIG. 5 is a series of photos of ZOI assays after 24 hours at 37° C., each comparing a lidocaine loaded band on the left and an off-the-shelf predicate band on the right, for (A) P. aeruginosa ATCC 15442, (B) E. coli ATCC 25922, (C) P. mirabilis ATCC 29906, (D) S. aureus ATCC 29213, (E) S. epidermidis ATCC 25984, (F) E. faecalis ATCC 51575, (G) C. albicans ATCC 10231.

FIG. 6 is a series of diagrams of ZOI quantitation, each measuring the outer diameter of a ZOI from FIG. 5 in two directions (left to right and top to bottom) and plotting the average of these, for (A) P. aeruginosa ATCC 15442, (B) E. coli ATCC 25922, (C) P. mirabilis ATCC 29906, (D) S. aureus ATCC 29213, (E) S. epidermidis ATCC 25984, (F) E. faecalis ATCC 51575, (G) C. albicans ATCC 10231.

FIG. 7 is a series of photos of planktonic inhibition assays showing turbidity of cultures after exposure to various elastrator bands for 24 hours for: (A) P. aeruginosa, (B) E. coli, (C) P. mirabilis, (D) S. aureus, (E) S. epidermidis, (F) E. faecalis, and (G) C. albicans.

FIG. 8 is a series of diagrams showing quantitation of planktonic cell density of each planktonic inhibition assay shown in FIG. 7 for: (A) P. aeruginosa, (B) E. coli, (C) P. mirabilis, (D) S. aureus, (E) S. epidermidis, (F) E. faecalis, and (G) C. albicans.

FIG. 9 is a graphic representation of the distribution of local anesthetic (active ingredient, “I”) throughout a drug-eluting medical device of the present disclosure in the exemplary form of an elastomeric band. The active ingredient (“I”) is infused and dispersed throughout the elastomeric band.

### DETAILED DESCRIPTION

The present disclosure relates to methods and devices for enhancing antimicrobial efficacy of a local anesthetic and in particular, using a drug-eluting medical device comprising the local anesthetic.

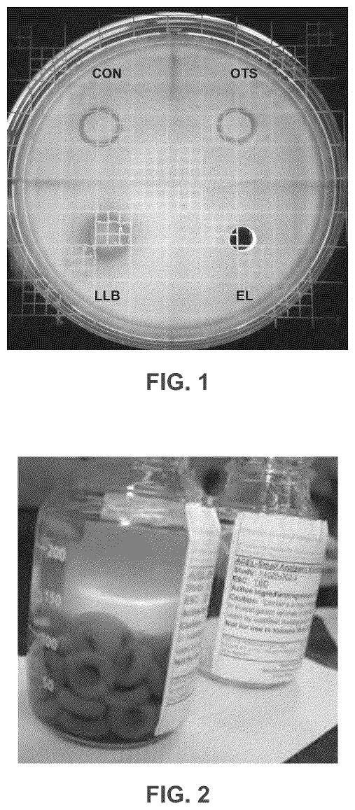

Advantageously, the drug-eluting medical device disclosed herein comprises the local anesthetic within an elastomeric material. As demonstrated herein, the local anesthetic is eluted from the drug-eluting medical device and the eluted local anesthetic provides an enhanced antimicrobial efficacy as compared to the same local anesthetic when not delivered by the drug-eluting medical device. In particular, as discussed below and shown in FIG. 1, the eluate containing the local anesthetic that is administered by elution from the drug-eluting medical device is shown to have an enhanced antimicrobial efficacy. Surprisingly, while the lidocaine loaded band (LLB) exhibited significantly enhanced antimicrobial efficacy as compared to the no lidocaine control (CON) and off-the-shelf (OTS) bands, the eluate from the LLB, when administered separately from the band, had no efficacy. This demonstrates that impregnation within, and delivery from the elastomeric band, is involved in the improvement in antimicrobial efficacy.

In some embodiments, the drug-eluting medical device further comprises a tissue permeator, a vasoconstrictor, and/or an antimicrobial agent within the elastomeric material.

### Methods

In an embodiment, the present disclosure relates to a method for enhancing antimicrobial efficacy of a local anesthetic, the method comprising: providing a drug-eluting medical device containing the local anesthetic, the drug eluting medical device comprising the local anesthetic within an elastomeric material; and administering the drug-eluting medical device to a subject such that it contacts a tissue and elutes the local anesthetic thereto; wherein the eluted local anesthetic provides an enhanced antimicrobial efficacy as compared to the same local anesthetic when not delivered by the drug-eluting medical device.

As used herein, by “enhancing antimicrobial efficacy” it is intended to mean (i) improving the antimicrobial activity of a substance that already has a baseline antimicrobial activity, and improving it beyond the baseline; and/or (ii) providing an antimicrobial activity to a substance that previously did not exhibit such activity. As used herein, “enhancing” may be by any number of means such as, without limitation, reducing the severity of infection, reducing the time course of infection, reducing the negative indications or health risks of infection, reducing any side effects associated with infection, improved killing of the microorganism, improved rapidity in neutralizing the microorganism, or any other form of therapeutic treatment or prevention of the infection caused by the microorganism.

The methods herein may be performed on any suitable subject. The subject may, for example, be a human or an animal (without limitation). In an embodiment, the subject is a human. In an embodiment, the subject is an animal. In an embodiment, the subject is a livestock animal. In an embodiment, the animal is cattle, goat, sheep, pig (swine), deer, elk, buffalo, bison, moose, alpaca, horse, donkey, zebus, yak, gayal, reindeer, or camel. In an embodiment, the animal is open-pasture reared. In an embodiment, the animal is containment reared (e.g. in a contained housing). In a particular embodiment, the animal is cattle, goat, sheep, deer or elk. In an embodiment, the drug-eluting medical device (e.g. elastomeric band) is affixed to the animal on or at a base of an animal part selected from a tail, a scrotum, a horn, an antler, a teat, an umbilicus, or a dermal growth (e.g. a cancer, a wart, etc.).

In an embodiment the methods herein involve affixing of the drug-eluting medical device (e.g. elastomeric band) proximate an existing wound site or an impending wound site for delivery of the local anesthetic for providing antimicrobial activity. In an embodiment, the existing wound site or the impending wound site is from castration, tail docking, de-antlering, umbilical cord ligation, or dehorning. In an embodiment, the existing wound site comprises microbial organisms or is at risk of developing infection by microbial organisms.

As used herein, the term “local anesthetic” refers to an anesthetic and/or analgesic that is administered directly to the site of action. The local anesthetic may refer herein to an individual compound or a composition that includes a local anesthetic and/or analgesic. In an embodiment, the local anesthetic is an amide-containing compound or an ester-containing compound. In an embodiment, the local anesthetic is a hydrophobic, a hydrophilic, or a lipophilic local anesthetic. Lipophilic anesthetics are compounds comprising at least one lipophilic group, such as for example an aromatic ring (e.g., benzene ring),

Examples of local anesthetics include, without limitation, lidocaine, free base lidocaine, bupivacaine, proparacaine, benzocaine, mepivacaine, bupivacaine liposomal, ropivacaine, chloroprocaine hydrochloride, levobupivacaine, and meloxicam. The lidocaine may, for example, be an acidic form or a free base form. In a preferred embodiment, the local anesthetic is lidocaine in free base form.

As used herein, the term “antimicrobial efficacy” refers to the ability and effect to prevent and treat infections by killing, or inhibiting growth of a microorganism, such as a bacteria, virus, fungus, or parasite. The term also includes and encompasses anti-biofilm efficacy or antibacterial efficacy. In an embodiment, the antimicrobial activity may be in respect of one or more (e.g. a panel) of wound-infecting microbial species including, but not limited to, a Gram-negative bacteria, a Gram-positive bacteria, a yeast, a virus, a fungus, a parasite, or any combination thereof.

In an embodiment, the enhancement in antimicrobial activity by the methods herein is in respect of a Gram-negative bacteria. In an embodiment, the enhancement in antimicrobial activity by the methods herein is in respect of a Gram-positive bacteria. In an embodiment, the enhancement in antimicrobial activity by the methods herein is in respect of a yeast. In an embodiment, the enhancement in antimicrobial activity by the methods herein is in respect of one or more of Pseudomonas aeruginosa, Escherichia coli, Proteus mirabilis, Staphylococcus aureus, Staphylococcus epidermidis, Enterococcus faecalis, Candida albicans, including any combination thereof. In an embodiment, the enhancement in antimicrobial activity by the methods herein in respect of one or any combination of these species is by lidocaine used in a drug-eluting medical device as disclosed herein, such as for example an elastomeric band. In an embodiment, the lidocaine is used in combination with a permeation enhancer, such as Dimethyl sulfoxide (DMSO), Isopropyl Myristate, n-methyl pyrrolidone (NMP), or any combination thereof. In an embodiment, the lidocaine or the lidocaine and permeation enhancer, is used in combination with a vasoconstrictor, such as those described herein.

The antimicrobial efficacy or potency can be measured, assessed, or determined by various appropriate testing methodologies. For example, a zone of inhibition (ZOI) assay or a Biofilm Surface Test Method (BSTM) may be used to quantitate antimicrobial activity. Furthermore, the antimicrobial efficacy or potency assessed may be expressed as a minimal inhibitory concentration (MIC), which is the lowest concentration of an antibiotic or antimicrobial agent needed to inhibit the growth of a given microbial species. Therefore, a lower MIC means enhanced potency.

As used herein, the term “drug-eluting medical device” refers to any medical device that contains a local anesthetic and is capable of eluting the local anesthetic, such as for example those described later herein (without limitation). In an embodiment, the medical device may be any appropriate and suitable device configured to be in contact with a subject's tissue, such as any epithelial tissue (e.g. skin), and may be of any suitable shape such as a ring, a band, a topical device, an implanted device, an indwelling medical device, or a urinary catheter. If a band, the band may be solid, hollow, porous, or tubular (e.g., defining a lumen). Where the band is tubular, the local anesthetic may be infused into the material, contained within the lumen of the tube or both infused into the material and contained within the lumen.

In an embodiment, the medical device is a transdermal patch. The transdermal patch may be of any suitable shape or configuration so as to elute the local anesthetic from the elastomeric material to the site of treatment. In an embodiment, the transdermal patch is an adhesive patch or a patch held in place by other means (e.g. Velcro).

In an embodiment, the medical device is an implant or prosthetic. The implant may for example be one that is used to deliver medication, monitor body functions, or provide support to organs and tissues. The prosthetic may be one that is intended to replace or supplement a missing or suboptimal functioning body part.

In an embodiment, the drug-eluting medical device is a catheter, such as a urinary catheter. The catheter may, for example, be an internal catheter, an external catheter, or a catheter that is both internal and external. The catheter may, for example, be an indwelling catheter, a condom catheter, or an intermittent self-catheter.

As used herein, the term “elastomeric material” refers to any pliable substance. Preferably, the elastomeric material is one suitable and medically appropriate for use in the drug-eluting medical device as described herein. In an embodiment, the elastomeric material is or comprises a rubber or polymeric material. The rubber may, for example, be any natural or synthetic rubber, or combination thereof. In other embodiments, the elastomeric material may be a silicone, a thermoplastic elastomer, or any combination thereof. For example, elastomeric materials include, but are not limited to, a natural rubber, a synthetic rubber, a silicone, a polybutadiene, a polyisoprene, a polychloroprene, a nitrile, a poly(styrene-butadiene-styrene) (SBS), a styrene-ethylene-butylene-styrene (SEBS), an ethylene-propylene-diene monomer rubber (EPDM), a polyurethane, a woven material, a natural fiber, a synthetic fiber, and any combination thereof. In an embodiment, the rubber or polymeric material is latex rubber.

In an embodiment, the drug eluting medical device of the present disclosure includes a local anesthetic within an elastomeric material. In such embodiments, it is contemplated herein that the local anesthetic is embedded within the elastomeric material, not merely coated onto the elastomeric material. The local anesthetic may be embedded within the elastomeric material by any suitable means. In an embodiment, the method of manufacture results in the local anesthetic being absorbed within the elastomeric material and embedded internally within the elastomeric structure of the drug eluting medical device. In an embodiment, the local anesthetic may be injected or impregnated into the elastomeric material. The local anesthetic is not merely coated on the elastomeric material, but this does not preclude the possibility that a minority portion of the local anesthetic may be found on the external surface of the elastomeric material, with the majority portion of the local anesthetic imbedded.

As used herein, by “providing” the drug-eluting medical device containing the local anesthetic, it is simply meant to make the drug-eluting medical device available for use in the methods disclosed herein. In an embodiment, the step of providing may include steps of preparing or manufacturing the drug-eluting medical device, such as in accordance with the present disclosure. In other embodiments, the drug-eluting medical device is pre-prepared and/or obtained from another source and “providing” merely refers to making it available for the “administering” step.

As used herein, the “administering” may be by any suitable means depending on the type of drug-eluting medical device that is used. In an embodiment, administering may be by affixing the drug-eluting medical device to a body part of the subject, such as an external body part. In an embodiment, administering may involve inserting the drug-eluting medical device into the subject, such as into a body opening or cavity. Various other forms of administering are contemplated herein and would be readily appreciated by the skilled person based on the type of drug-eluting medical device that is used.

As used herein, the term “subject” refers to any living mammal to which the drug-eluting medical device may suitably be administered. For example, in an embodiment, the subject is a human. In an embodiment, the subject is a livestock animal, such as for example cattle, goat, sheep, pig, deer, elk, buffalo, bison, moose, alpaca, horse, donkey, zebus, yak, gayal, reindeer, or camel.

As used herein, the term “tissue” refers to any tissue of the subject. For example, a tissue includes, but is not limited to, an epithelial tissue. The tissue may be one that is internal or external on the body of the subject. In an embodiment, the tissue is epithelial tissue, such as for example skin, the lining of intestines, the lining of the respiratory tract, the lining of blood vessels, the lining of the abdominal cavity, periodontium, gingiva, or any other epithelial tissue. In a particular embodiment, the epithelial tissue is the outer layer of skin.

As used herein, the term “elute” refers to the process where the local anesthetic is released from the drug-eluting medical device. For example, elute includes, but is not limited to, releasing, delivering, or leaching. The local anesthetic may elute from the elastomeric material at different rates depending on the type of local anesthetic used, the type of elastomeric material used, and the manner in which the local anesthetic is embedded within the elastomeric material. Moreover, the rate of elution may be adjusted as desired based on one or more of these characteristics.

As shown in FIG. 1 herein, the eluted local anesthetic has been shown to have improved antimicrobial activity when associated with the elastomeric band (see LLB), with a zone of inhibition extending outwards from the band. In contrast, no antimicrobial activity was observed for the no lidocaine “mock processed” control (CON), the off-the-shelf control (OTS), and the aqueous eluate from the LLB (EL). This demonstrates that the improved efficacy is limited to the association of the local anesthetic with the band at the time of administration (i.e. being delivered from the band).

In an embodiment, the drug-eluting medical device further contains a tissue permeator within the elastomeric material. In an embodiment, the tissue permeator is isopropyl myristate, fatty acids, fatty acid esters, poloxamers, triglycerides, n-methyl pyrrolidone, terpineol, limonene, dimethyl sulfoxide, dimethylacetamide, or any combinations thereof.

In an embodiment, the drug-eluting medical device further contains a vasoconstrictor within the elastomeric material. In an embodiment, the vasoconstrictor is epinephrine, pseudoephedrine, phenylephrine, thromboxane, or angiotensin.

In an embodiment, the drug-eluting medical device further contains an antimicrobial agent within the elastomeric material. The antimicrobial agent may, for example, be an alcohol, a cationic, surface-active quaternary ammonium compound, a bisphenol, a chloride compound, an iodine or iodized compound, an aldehyde, a mercurial, an oligodynamic metal, a heavy metal, an acid, a dye, an antibiotic drug, a chemotherapeutic drug, a coal-tar additive, an aromatic oil, or any other suitable agent or compound having antimicrobial activity. In an embodiment, the antimicrobial agent is high valency silver or silver sulphadiazine.

An advantageous aspect of the method the present disclosure is that the antimicrobial efficacy of the local anesthetic is enhanced, compared to the same local anesthetic when not delivered by the drug-eluting medical device. For example, the antimicrobial efficacy is improved as compared to delivery of the local anesthetic merely coated on the surface of a medical device. For example, the antimicrobial efficacy is improved as compared to delivery of the local anesthetic in free form (not using a medical device).

According to one embodiment of the present disclosure, a lidocaine loaded band (LLB) is created and manufactured as the anesthetic/antimicrobial eluting medical device. Then, appropriate methodologies are also developed to investigate the ability of LLBs to elute substances with antimicrobial and/or anti-biofilm efficacy, as described below in detail. As used herein, the term “lidocaine loaded band” may refer, as an example, to an elastomeric band as described herein that is infused with the lidocaine.

In particular, the ability of LLBs to inhibit the growth of representative Gram-negative and Gram-positive bacteria (Escherichia coli and Staphylococcus aureus, respectively) by a zone of inhibition (ZOI) assay was assessed, which aimed to simulate elution of lidocaine out of the device and into a contacted tissue.

Next, the potential antimicrobial and anti-biofilm activities of LLBs against a small panel of wound-infecting microbial species, including Gram-negative and Gram-positive bacteria, was quantitated by log₁₀ CFU reduction. The protocol used herein was based on an ASTM standard practice, currently in development, called the Biofilm Surface Test Practice (BSTP). The BSTP is designed to quantitate the ability of antimicrobial impregnated medical devices to resist biofilm formation, as well as to inhibit planktonic microbial growth via leaching.

Next, an agar-based ZOI assay and a broth-based log₁₀ reduction assay are expanded to a broader panel of wound-infecting microbial species, including three Gram-negative bacteria (Pseudomonas aeruginosa, E. coli, and Proteus mirabilis), three Gram-positive bacteria (S. aureus, Staphylococcus epidermidis, and vancomycin-resistant Enterococcus faecalis or “VRE”), and one yeast (Candida albicans).

An initial assay demonstrated that lidocaine-loaded bands (LLBs) elute an antimicrobial agent, producing a zone of inhibition (ZOI) on lawns of Escherichia coli and Staphylococcus aureus and indicating that this antimicrobial activity can be successfully delivered into a semi-solid agar, simulating delivery into tissue.

A follow-up assay indicated that lidocaine elution from LLBs into a serum-spiked broth significantly inhibited growth of Pseudomonas aeruginosa and was a potent bactericide against E. coli and S. aureus. Moreover, similar efficacy was observed against biofilms of the aforementioned organisms.

Next, as the agar-based ZOI assay and the broth-based log₁₀ reduction assay are expanded to a broader panel of wound-infecting microbial species, including several Gram-negative and Gram-positive bacteria and one yeast, several observations were made and conclusions were drawn.

First, no ZOI was formed for the “off-the-shelf” (i.e., predicate) latex rings for any of the tested organisms, while significant ZOIs (of varying diameters) were formed by LLBs for all seven organisms. This confirms that LLBs elute an antimicrobial agent(s) with at least some efficacy against a very broad spectrum of wound-infecting microorganisms.

Second, relative to the predicate controls, the mock-processed controls (containing excipients tetrahydrofuran (THF) and isopropyl myristate (IPM) but no Lidocaine) only yielded statistically significant (P<0.05) log₁₀ reductions >1 for S. epidermidis. In contrast, the LLBs yielded statistically significant log₁₀ reductions >1, relative to either the mock processed controls or the predicate controls, for all seven tested organisms. Together, these data indicate that the processed bands lack a significant antimicrobial effect in the absence of lidocaine, although it is possible that lidocaine still requires one of the other components (IPM or THF) for full potency.

LLBs yielded log₁₀ reductions, relative to the predicate control bands, of >9.7 (“max kill”), >7.6 (“max kill”), 5.4, 4.3, 2.3, 2.1, and 1.4 for E. coli, C. albicans, S. aureus, S. epidermidis, P. aeruginosa, VRE, and P. mirabilis, respectively.

Notably, each organism had a starting inoculum density of ˜10⁶ CFU/mL, which climbed over the 24-hour incubation period to ˜10⁹ (for the bacteria) or 10⁷ (for the yeast) in the predicate control wells; however, the LLBs reduced this density to ˜10⁴, ˜10⁴, 0, and 0 for S. aureus, S. epidermidis, E. coli, and C. albicans, respectively, indicating that LLBs elute a microbicidal agent for these four organisms. LLBs may only elute an inhibitory agent for P. aeruginosa, P. mirabilis, and E. faecalis.

Third, broth microdilution was used to assess the relative potency of various reagents, in the context of microbial growth medium rather than eluted from an LLB, expressed as the minimal inhibitory concentration (MIC).

Because lidocaine-HCl is water soluble, a range of relatively high concentrations could be tested as a positive control. Indeed, the MIC could be observed for all seven tested organisms. The MIC for lidocaine-HCl was 25.0, 6.25, 12.5, 12.5, 25.0, 25.0, and 12.5 mg/mL for P. aeruginosa, E. coli, P. mirabilis, S. aureus, S. epidermidis, E. faecalis, and C. albicans, respectively. This is generally consistent with published literature, which indicates that the MIC for lidocaine-HCl is >20.0, 5.00, and 20.0 mg/mL for P. aeruginosa ATCC 27853, E. coli ATCC 25922, and S. aureus ATCC 29213, respectively.

Unlike lidocaine-HCl, free base lidocaine (which is used in LLBs) has limited aqueous solubility, limiting the maximum challenge concentration to 1 mg/mL. All organisms grew at this highest tested concentration of lidocaine base, indicating that the MIC is greater than 1 mg/mL for all seven tested organisms.

Although the tissue permeator IPM is immiscible with microbial growth broth, two alternative permeation enhancers, Dimethyl sulfoxide (DMSO) and n-methyl pyrrolidone (NMP), were each tested at a maximum challenge concentration of 4% v/v. All seven organisms grew at the maximum concentration of DMSO, indicating an MIC of >4% v/v for DMSO. In contrast, all seven organisms were inhibited at the highest concentration of NMP, indicating an MIC =4% v/v for NMP.

To test whether either permeation enhancer displayed synergy with lidocaine base (i.e., had a lower MIC, indicating enhanced potency), the titrations were performed in the presence of a constant concentration of 1 mg/mL lidocaine base. Again, the MIC for DMSO (plus lidocaine base) or NMP (plus lidocaine base) was >4 and =4% v/v, respectively, indicating no measurable synergy between lidocaine base and these two IPM surrogates.

Fourth, the levels of IPM and THE eluted from the bands into the growth medium in the planktonic inhibition assay (see second point above) were all below the limit of quantitation (LoQ), while the level of free-base lidocaine eluted from the bands into the growth medium averaged 2.9 mg/mL, which was substantially lower (between 2.2-and 8.6-fold lower) than the MICs for lidocaine-HCl reported above (see above). This suggests some enhancement of lidocaine's antimicrobial efficacy in the context of the LLBs.

The enhanced antimicrobial activity of the lidocaine (as demonstrated by the lower MIC) when incorporated into the medical device is an unexpected discovery. Without being bound by theory, this may be due to the increased lipophilic character of the lidocaine free-base relative to lidocaine-HCl, or due to synergy with one or more excipients present in the LLB, e.g. that is only effective when the lidocaine is administered by the LLB (e.g., IPM or residual THF solvent).

Taken above points together, overall, LLBs possess antimicrobial activity against a broad range of wound-infecting bacteria, with a greater potency than would be expected for lidocaine alone. This discovery is thus valuable for the prevention of infection along with provision of local anaesthesia when incorporated into a medical device or similar tissue contacting surface.

Turning now to FIG. 1, which is a photo of a zone of inhibition (ZOI) schematic for E. coli, American Type Culture Collection (ATCC) 25922, showing ZOI of a lidocaine loaded band (LLB); a no lidocaine “mock processed” control (CON); an off-the-shelf control (OTS); and an aqueous eluate from a LLB (EL).

E. coli (ATCC 25922) were grown in tryptic soy broth (TSB) to saturation (˜9 log₁₀ CFU/mL), diluted 1000-fold in molten TSA (maintained at 50° C.), and the resulting suspension poured onto TSA base plates. After solidifying (˜3 minutes), the well evident in the “EL” quadrant was bored with a 6-mm biopsy punch and filled with 100 μL of LLB eluate (i.e., phosphate buffered saline (PBS) in which LLBs had been soaked for 24 hours at 37° C.). The EO-sterilized rings were placed, as indicated, with sterile forceps. The plates were then sealed in sandwich bags and incubated for 96 hours at 37° C. “LLB” refers to lidocaine loaded band. “CON” refers to no lidocaine “mock processed” control. “OTS” refers to off-the-shelf control. “EL” refers to LLB liquid eluate.

In particular, a comparison between EL and LLB shows a difference. As shown in FIG. 1, the area labelled EL has a hole but no zone of inhibition beyond. This thus indicates that the eluate has no antimicrobial efficacy compared to the LLB. No zone of inhibition was evident for the CON and the OTS.

Details of the testing methods and results are described below.

### Manufacturing Methods

Exemplary manufacturing methods are described below. These are intended as non-limiting and exemplary.

In an embodiment, latex rings were soaked for 180 minutes at room temperature (approx. 20° C.-30° C.) in a solution containing:
a. Free-base lidocaine: 5-40% w/w
b. Isopropyl Myristate (IPM): 10-35% w/w
c. Ethanol: 1-10% w/w
d. De-ionized water: 0.1-1.0% w/w
e. Tetrahydrofuran: 30-75% w/w

During soaking, bands were impregnated with the free-base lidocaine and IPM.

FIG. 2 is a photo showing bands soaking.

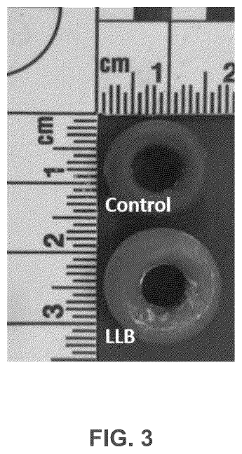

FIG. 3 is a photo showing a comparison of size between a control band (off-the-shelf control) and a finished LLB that is swollen. The finished LLB has a larger diameter than the control band.

Bands were then dried to remove the solvent (e.g., air dried at room temperature for >48 hours, or dried in an oven at 60±1° C. for approx. 30 minutes). Yields bands were approximately 15.6-mm outer diameter, 3.6-mm inner diameter, 6.0-mm thick, and weighing 713 mg.

First, the yields bands contain free-base lidocaine (API), approximately 85 mg per band. This API acts both as a local anesthetic and an antimicrobial, the latter likely involves damage to the cell membrane or envelope and subsequent leakage of intracellular components, dehydrogenase activity, and increased cell wall permeability. Relative to lidocaine-HCl, free-base lidocaine is lipophilic (important for incorporation into latex as well as permeation of tissue and microbial cells; lower water solubility yields prolonged activity).

Second, the yields bands contain IPM (a tissue permeator), approximately 110 mg/band. This provides improved tissue penetration by the free-base lidocaine and may also improve microbial penetration and/or yield a synergistic effect with lidocaine in terms of cell wall or membrane permeability.

Third, the yields bands contain THF (a residual solvent), <28 mg/band.

The lidocaine-loaded bands (LLB) deliver the API into the contacted tissue over the course of a catheterization (or similar) procedure, affecting local anesthesia and anti-microbial and anti-biofilm activity. This product thus delivers both reduced local sensation during painful procedures, and infection treatment/prophylaxis.

### Drug-Eluting Medical Devices

In an embodiment, the present disclosure relates to a drug-eluting medical device providing both anesthetic and antimicrobial effects, the drug-eluting medical device comprising a local anesthetic contained within an elastomeric material from which it is eluted.

As shown herein, drug-eluting medical devices of the present disclosure are capable of enhancing and/or improving antimicrobial efficacy as compared to the same local anesthetic when not delivered by the drug-eluting medical device.

In an embodiment, the ring or band comprises or consists of an elastomeric material having the local anesthetic infused within the elastomeric material. In an embodiment, the elastomeric material is a rubber or polymeric material. The rubber or polymeric material may, for example, be selected from a natural rubber, a synthetic rubber, a silicone, a polybutadiene, a polyisoprene, a polychloroprene, a nitrile, a poly(styrene-butadiene-styrene) (SBS), a styrene-ethylene-butylene-styrene (SEBS), an ethylene-propylene-diene monomer rubber (EPDM), a polyurethane, a woven material, a natural fiber, a synthetic fiber, and any combination thereof. In an embodiment, the rubber or polymeric material is latex rubber.

In an embodiment, the drug-eluting medical device is a tube, ring or a band, such as for example and without limitation the ligation device as disclosed in International Patent Publication No. WO 2019/032928, published on Feb. 14, 2019, which is incorporated by reference herein in its entirety. In other embodiments, and again without limitation, the drug-eluting medical device may be of similar composition and/or construction as the elastomeric band described in International Patent Application No. PCT/CA2024/050216 (not yet published), which is incorporated by reference herein in its entirety.

In an embodiment, the drug-eluting medical device advantageously provides release of the local anesthetic by both a fast-acting mode and a slow-acting mode, wherein: during the fast-acting mode, a first portion of the local anesthetic is delivered to a body surface of a subject and/or an environment in close proximity to the body surface, immediately upon affixing the drug-eluting medical device (e.g. elastomeric band) to the subject; and during the slow-releasing mode, a second portion of the local anesthetic is delivered to the body surface of the subject and/or the environment in close proximity to the body surface, gradually over a period of time after continued affixation of the drug-eluting medical device (e.g. elastomeric band) to the subject. Such advantageous effects are described in relation to insecticides in International Patent Application No. PCT/CA2024/050216 (not yet published), and are equally applicable to the delivery of the local anesthetics by the drug-eluting medical device disclosed herein taking the form of an elastomeric band.

In an embodiment of the drug-eluting medical device (e.g. elastomeric band), only a portion of the drug-eluting medical device (e.g. elastomeric band) comprises the local anesthetic, the portion being a defined and selective region or zone of active agent. Various methods for preparing the drug-eluting medical device (e.g. elastomeric band) may be used, such as for example as described in International Patent Publication No. WO 2019/032928 and/or International Patent Application No. PCT/CA2024/050216 (not yet published), both of which are incorporated by reference herein in its entirety.

Exemplary drug-eluting medical devices (e.g. elastomeric bands) of the present application may be prepared in various sizes (e.g. small band format, medium band format, or large band format). In an embodiment, local anesthetic may be infused into the entirety of the band for non-selective, total device infusion. In another embodiment, local anesthetic may be infused into only a portion of the band for selective, targeted infusion.

Generally, drug-eluting medical devices (e.g. elastomeric bands) of the desired size (small, medium and large) may be obtained from a commercial source. Bands may be submersed in their entirety in a solution containing a local anesthetic. In other embodiments, bands may be prepared by submersing only a portion of the band in a solution containing the local anesthetic. A colored dye may be included in the solution to visually observe the localized infusion of the solution into the band. Submersing only a portion of the band in the solution can be effective in providing a band with a defined and selective region or zone of active agent. Advantageously, the manufacture of bands in this manner provides a region or zone that is free of local anesthetic that may be used for safe handling of the bands. This is not only advantageous in respect of ease of handling, but also reduces waste since rubber gloves to not need to be worn when handling the bands. This is disclosed in International Patent Application No. PCT/CA2024/050216 (not yet published) in respect of insecticides, and may be similarly employed in relation to local anesthetics of the present disclosure.

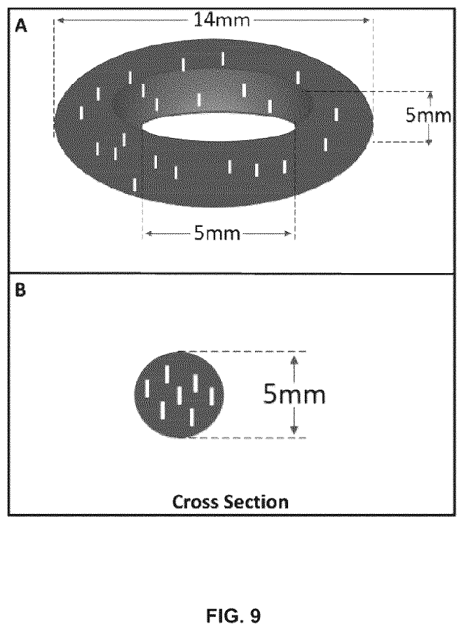

FIG. 9 shows a graphic representation of the distribution of active ingredient (“I”) throughout the band. As shown, the local anesthetic can be infused and dispersed throughout the band. Notably, bands containing different active ingredients, or combinations thereof, may can be color-coded by using band materials of different color (e.g. orange, green, blue, black, yellow, etc.). Advantageously, by using elastomeric bands, it has been found that after absorption of the active ingredient into the elastomeric material, shrinking of the band upon drying will cause some of the active ingredient contained within the band to become disposed on the surface of the band, thus improving the fast-acting mode of release as described herein. This is equally applicable to the release of a local anesthetic from the drug-eluting medical devices disclosed herein in the enhancement of antimicrobial efficacy of the local anesthetic.

In an embodiment, the drug-eluting medical device is a topical, implanted, or indwelling medical device.

In an embodiment, the drug-eluting medical device is a catheter, such as a urinary catheter. The catheter may, for example, be an internal catheter, an external catheter, or a catheter that is both internal and external. The catheter may, for example, be an indwelling catheter, a condom catheter, or an intermittent self-catheter.

In an embodiment, the drug-eluting medical device further comprises a tissue permeator within the elastomeric material. The tissue permeator may, for example, be isopropyl myristate, fatty acids, fatty acid esters, poloxamers, triglycerides, n-methyl pyrrolidone, terpineol, limonene, dimethyl sulfoxide, dimethylacetamide, or any combination thereof.

In an embodiment, the drug-eluting medical device further contains a vasoconstrictor within the elastomeric material. The vasoconstrictor may, for example, be epinephrine, pseudoephedrine, phenylephrine, thromboxane, or angiotensin.

In an embodiment, the drug-eluting medical device further contains an antimicrobial agent within the elastomeric material. The antimicrobial agent may, for example, be an alcohol, a cationic, surface-active quaternary ammonium compound, a bisphenol, a chloride compound, an iodine or iodized compound, an aldehyde, a mercurial, an oligodynamic metal, a heavy metal, an acid, a dye, an antibiotic drug, a chemotherapeutic drug, a coal-tar additive, an aromatic oil, or any other suitable agent or compound having antimicrobial activity. In an embodiment, the antimicrobial agent is high valency silver or silver sulphadiazine.

### EXAMPLES

### Experimental Test Matrices

(1) ZOI:
a. Two (2) Test Articles per plate: LLB and “Off-The-Shelf” Predicate Rings;
b. Seven (7) test organisms (3 Gram-positive, 3 Gram-negative, 1 yeast);
c. Triplicate (n=3).
d. Total=42 individual samples on 21 agar plates.

(2) Log₁₀ Reduction:
a. Three (3) Test Articles per plate: LLB, “Mock Processed” Controls, and “Off-The-Shelf” Predicate Rings;
b. Seven (7) test organisms (3 Gram-positive, 3 Gram-negative, 1 yeast);
c. Triplicate (n=3), plus one sterility control per band type.
d. Total=84 individual samples on 7 multi-well plates.

### Objectives of the Study

The purpose of this study was to test whether LLBs possess antimicrobial properties that can leach/elute into a contacted agar matrix to form a ZOI against lawns of a variety of wound-associated microbial species; and leach/elute into a simulated wound exudate broth to inhibit planktonic growth of the same organisms.

A secondary objective was to determine the minimal inhibitory concentration (MIC) of free-base lidocaine, permeation enhancers DMSO or NMP, or their combinations, by broth microdilution against the same panel of organisms.

A tertiary objective was to determine the amount of lidocaine (LD), THE, and IPM eluted into broth during the planktonic growth inhibition assay.

### Sample Description

TABLE 1
Sample Tested

| CODE | SAMPLE | Description | Lot |
|---|---|---|---|
| LLB | LD-Loaded Band | 4.3-cm circumference, | ADEL-22-G-003 |
| | | 0.5-g green elastrator | |
| | | band. Contains about 78 | |
| | | mg of lidocaine per | |
| CON | No LD Control | “Mock processed” (i.e., | ADEL-21-G-005 |
| | Band | same as LLB but no LD | |
| OTS | Off-the-Shelf | Unprocessed Predicate | N/A |
| | | bands (master control). | |
| LD-HCl | Lidocaine HCl | Lidocaine hydrochloride | 171585/B |
| | | for MIC test. | |
| LD-B | Lidocaine Base | Free base lidocaine for | MC/LB/790/14 |
| | | MIC test. | |
| DMSO | Dimethyl | Permeation enhancer for | AD-20325 |
| | Sulfoxide | MIC test. | |
| NMP | n-methyl-2- | Permeation enhancer for | CL2 |
| | pyrrolidone | MIC test. | |

a. Contact Times: 24 hours (ZOI formation was monitored for up to 6 days).
b. Contact Temperature: 37±1° C.
c. Biofilm Growth Medium (BFG): Mixed 100 mL sterile organism specific broth (OSB) with 100 mL heat-inactivated sterile fetal bovine serum and 300 mL sterile PBS, pH 7.4.
d. Growth Surfaces: CellStar 12-well plates (Greiner Bio-One) were used for planktonic inhibition experiments. Nunclon Delta polystyrene 96-well flat bottom plates were used for microdilutions. TSA plates were used for ZOI experiments.

### Microorganisms

TABLE 2
Microorganisms

| | | | Storage | | Risk |
|---|---|---|---|---|---|
| # | Organism | Source/Strain | Location | OSB/OSA | Group* |
| 1 | Pseudomonas | ATCC 15442 | 6:77 | TSB/TSA | 2 |
| | aeruginosa | | | | |
| 2 | Escherichia | ATCC 25922 | 6:73 | TSB/TSA | 1 |
| | coli | | | | |
| 3 | Proteus | ATCC 29906 | 7:6 | TSB/TSA | 2 |
| | mirabilis | | | | |
| 4 | Staphylococcus | ATCC 29213 | 6:76 | TSB/TSA | 2 |
| | aureus | | | | |
| 5 | Staphylococcus | ATCC 35984 | 1:22 | TSB/TSA | 1 |
| | epidermidis | | | | |
| 6 | Enterococcus | ATCC 51575 | 1:61 | TSB/TSA | 2 |
| | faecalis | | | | |
| 7 | Candida | ATCC 10231 | 2:42 | TSB/TSA | 1 |
| | albicans | | | | |

a. BSL2 precautions were taken.
b. Culture was grown and challenged in TSB/TSA at 37° C.
c. #1-3 are Gram-negative bacteria; #4-6 are Gram-positive bacteria; and Candida albicans is yeast.

### Test Method Overview

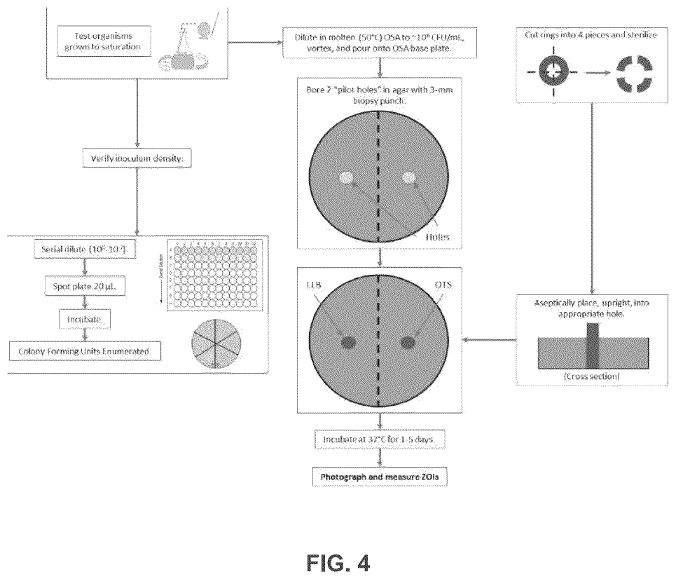

ZOI Assay: Each test organism was grown to saturation in OSB (37° C., 150 rpm, 24 hours). These saturated overnight (“O/N”) cultures were diluted to ˜10⁶ CFU/mL in molten TSA (maintained at 50° C.) and poured onto a TSA base plate. Each test article was placed on the agar plates according to FIG. 4 using sterile forceps. Plates were then incubated at 37° C. until a suitable endpoint was reached (not more than 6 days), at which point photographs were taken and the diameter of the inhibition zones carefully measured.

Log Reduction Assay: Each test organism was grown to saturation as above, diluted to ˜10⁶ CFU/mL in biofilm growth medium (BGM), and 4 mL of this inoculum placed into the wells of a 12-well plate. The various bands were placed into the appropriate wells and the plates incubated (37° C., 110 rpm) for 24 hours. Finally, samples from each well were diluted, plated, and the viable cells enumerated. The log reduction in viable cells was calculated by subtracting the mean log₁₀ density for the impregnated devices from the log₁₀ density of the untreated (control) devices.

Broth Microdilution Assay: A stock solution of each test article was serially diluted in a 96-well plate for a final volume of 0.1 mL per well. Each test organism was grown to saturation as above, diluted to ˜10⁵ CFU/mL in BGM, and 0.1 mL added to each well of the 96-well plate. Turbidity was assessed after 16-24 hours at 37° C. (100 rpm).

Turning now to FIG. 4, which is a flow diagram representing the experimental process for the ZOI antimicrobial assay. This protocol may be broken into a series of small steps, each of which is detailed in the sections below.

### Culture/Inoculum Preparation

Using a cryogenic stock (at −70° C.), streaked out a first sub-culture of the organisms listed above (see above section MICROORGANISMS) on organism specific agar (OSA).

Incubated at 37±1° C. for 16-24 hours and stored the plate wrapped in parafilm at 4° C. From the first sub-culture, streaked out a second sub-culture on OSA. Incubated at 37±1° C. for 16-24 hours. The second sub-culture was used within 24 hours starting from the time it was first removed from incubation.

Using the second sub-culture, aseptically removed 1-5 isolated colonies from the OSA plate and inoculated 5 mL of OSB in a 50-mL screw-top tube (or similar).

Placed the culture on an orbital shaker in a humidified incubator; incubated at 200 rpm at 37±1° C. for 24 hours. This yielded a culture of approximately 10⁹ CFU/mL for the bacteria and 10⁷ CFU/mL for the yeast.

Moved 100 μL of the saturated O/N culture into each of two wells (in row A) of a 96-well plate for an O/N inoculum check:
a. Added 180 μL of sterile 0.9% saline to the remaining rows.
b. Serially diluted with a multichannel pipettor (10⁰-10⁻⁷) by transferring 20 μL down each of the 8 rows for each plate. Mixed the contents by pipetting up and down at least 3 times. After mixing each row, discarded the pipette tips. Using fresh tips, continued the dilution procedure.
c. Using a single channel pipettor, removed 20 μL from each well and plate on OSA plates. Used a fresh tip for each well. NOTE: One OSA plate was used for each dilution series by dividing individual plates into eight pie-shaped sections and labelling the sections 10⁰-10⁻⁷. Incubated the agar plates at 37±1° C. for 16-24 hours and enumerated colonies.

### Test Article Preparation

Placed a sufficient number of test and control rings (>28 of each) into an appropriate sterilization pouch, labelled, and sealed.

Cut >6 test and >6 control rings into 4 equal pieces (producing >24 pieces of each); placed into a pouch, labelled, and sealed.

Sterilized with ethylene oxide.

### Preparation of Bacterial Lawns and Challenge with Test Articles

Using a magnetic stir bar, dissolved 10.0 g of tryticase soy agar (TSA) powder in 250 mL of deionized water on a stir plate heated to ˜95° C.

Ensured that a sufficient vortex was achieved such that the powder remained evenly suspended; while stirring, used a serological pipette to remove 10 mL to each of 25 glass screw-top tubes.

With the lids slightly loosened, autoclaved the tubes. When the cycle was complete, gently tightened the lids and placed the tubes in a 50° C. water bath to maintain the agar in its molten state.

The above steps were conducted the afternoon prior to the challenge (see below), and the tubes were held at 50° C. to maintain the agar in its molten state.

Using an appropriate marker, divided each of the 21 OSA base plates into quadrants and labelled appropriately.

The day of the challenge (see below), dilute the saturated bacterial O/N culture 1:1,000 (i.e., 10 μL of O/N into each tube of 10 mL molten TSA); diluted the yeast culture 1:10 (i.e., 1 mL plus 9 mL molten TSA).

Immediately vortexed the tube for at least 5 seconds to ensure an even suspension of the bacterial cells and poured the contents of the tube onto an agar base plate. Repeated for the other 20 base plates; the remaining TSA tubes were spares. NOTE: Worked quickly and only inoculated 1 tube at a time, to ensure that the TSA did not solidify before it could be properly mixed and poured onto the base plate.

Allowed the inoculated TSA to solidify on the base plates (about 3-5 minutes at RT).

Used a sterile 3-mm biopsy punch to bore two holes into each agar plate (according to FIG. 4).

Opened the sterile packages of the test articles.

Using sterile forceps/tweezers, inserted the cut pieces of each test article into the appropriate hole of each plate (per FIG. 1).

Sealed the plates, upright, in sandwich bags and incubated at 37±1° C. until a suitable endpoint was reached (i.e., formation of an obvious ZOI, or until a maximum incubation time of 6 days, whichever occurred sooner).

Photographed each plate. Using calipers, carefully measured the outer diameter for each ZOI, in each of two directions (Left to Right and Top to Bottom) and recorded.

### Planktonic Growth/Challenge and Log Reduction Assay

Diluted the culture from above in Biofilm Growth Medium (BGM), for an approximate inoculum density of 10⁶ CFU/mL. (For bacteria: 40 μL O/N plus 40 mL BGM; for yeast: 4 mL O/N plus 36 mL BGM). Vortexed the diluted sample for approximately 5 seconds to achieve a homogeneous distribution of the cells.

Aliquoted 4 mL of the inoculum to each well of columns 1-3 of the 12-well plate, and 4 mL of sterile BGM into each well of column 4 to serve as sterility controls (see below Table). Placed the LLB, OTS, or CON bands into the appropriate wells (see below Table 3).

Placed the lid onto the plate, labelled the plates appropriately, and sealed in a sandwich bag.

Placed the plates on the orbital shaker in a humidified incubator. Set shaker to 110 rpm to prevent spill-over. Incubated at 37±1° C. for 24 hours.

After 24 hours of incubation, removed 100 μL of inoculum from each well to a well in row A of a 96-well plate (one 96-well plate per 12-well plate), serially diluted from 10⁰ to 10⁻⁷, and spot plated to check planktonic density, as described in above step under CULTURE/INOCULUM PREPARATION.

Turbidity of the inoculum was visually assessed and photographed at this point.

TABLE 3
Test plate layout for Planktonic Inhibition

| | 1 | 2 | 3 | 4 |
|---|---|---|---|---|
| A | LLB | LLB | LLB | LLB (SC) |
| B | CON | CON | CON | CON (SC) |
| C | OTS | OTS | OTS | OTS (SC) |

a. “LLB” refers to the lidocaine-loaded bands, “CON” refers to the “mock processed” control bands;
b. “OTS” refers to the off-the-shelf predicate control bands; and
c. “SC” refers to their respective sterility controls (i.e., uninoculated wells). Each plate represents one test organism (total=7 test plates).

### Broth Microdilution of Lidocaine

Prepared a 2× stock solution of lidocaine by dissolving 20 mg of free-base lidocaine per 10 mL of OSB (=2 mg/mL). Incubated at 37° C. at 200 rpm for approximately 2 hours to fully dissolve the powder. Filter sterilized.

Prepared a 2× stock of lidocaine HCl by dissolving 500 mg per 10 mL OSB (=50 mg/mL). Filter sterilized.

Prepared 2× stock (=8% v/v) of DMSO or NMP by mixing 800 μL of either with 9.2 mL of OSB or with OSB plus 2 mg/mL lidocaine base.

Aliquoted 100 μL of OSB into each well of the first row of seven 96-well plates to serve as positive growth controls (see below Table 4).

Aliquoted 200 μL of the 2× stock of LD-HCl, LD base, DMSO, NMP, DMSO+LD base, or NMP+LD base into wells B1-2, B3-4, B5-6, B7-8, B9-10, or B11-12, respectively.

Aliquoted 100 μL of the 2× stock of LD-HCl, LD base, DMSO, NMP, DMSO+LD base, or NMP+LD base into wells H1-2, H3-4, H5-6, H7-8, H9-10, or H11-12, respectively. These served as the sterility controls.

Aliquoted 100 μL of OSB into each well of rows C to G, columns 1-8.

Aliquoted 100 μL of OSB+2 mg/mL LD base into each well of rows C to G, columns 9-12.

Serially diluted (2-fold series) the contents of rows B to G: Removed 100 μL from row B to row C and mixed by pipetting up and down at least 3 times. Discarded the tips.

Using fresh tips, repeated for rows C to D, and so on. Did not proceed past row G.

After mixing row G, removed and discarded 100 μL from row G, yielding 100 μL of liquid in all wells.

Diluted the O/N culture (from above step under CULTURE/INOCULUM PREPARATION) in BGM for an approximate inoculum density of 10⁵ CFU/mL:
a. For bacteria: Mixed 10 μL O/N with 990 μL OSB for ˜10⁷ CFU/mL, then mixed 100 μL of the ˜10⁷ CFU/mL culture with 9.90 mL BGM for ˜10⁵ CFU/mL.
b. For yeast: Mixed 100 μL O/N with 9.90 mL BGM for ˜10⁵ CFU/mL. Vortexed the diluted sample for approximately 5 seconds to achieve a homogeneous distribution of the cells.

Immediately poured the ˜10⁵ CFU/mL inoculum into a sterile reagent reservoir and used a multichannel pipettor to aliquot 100 μL into each well of the 96-well plates, excluding row H. Mixed by pipetting up and down at least 3 times. Added uninoculated BGM to row H.

Sealed the plates in sandwich bags and incubated at 37±1° C. for 16-24 hours (100 rpm).

Visually scored for growth, which appeared as turbidity or as a deposit of cells at the bottom of a well.

Determined the MIC (minimum inhibitory concentration) for each organism. The MIC was defined as the minimum concentration that inhibited growth of the organism.

TABLE 4
Broth Microdilution Plate Layout

| | | | | | | | | | 9 | 10 | | |
|---|---|---|---|---|---|---|---|---|---|---|---|---|
| | 1 | 2 | 3 | 4 | 5 | 6 | 7 | 8 | DMSO | | 11 | 12 |
| | LD-HCl | | LD-B | | DMSO | | NMP | | (+1 mg/mL LD) | | NMP | |
| A | GC | GC | GC | GC | GC | GC | GC | GC | GC | GC | GC | GC |
| B | 25.0 | 25.0 | 1.00 | 1.00 | 4.00 | 4.00 | 4.00 | 4.00 | 4.00 | 4.00 | 4.00 | 4.00 |
| C | 12.5 | 12.5 | 0.50 | 0.50 | 2.00 | 2.00 | 2.00 | 2.00 | 2.00 | 2.00 | 2.00 | 2.00 |
| D | 6.25 | 6.25 | 0.25 | 0.25 | 1.00 | 1.00 | 1.00 | 1.00 | 1.00 | 1.00 | 1.00 | 1.00 |
| E | 3.12 | 3.12 | 0.12 | 0.12 | 0.50 | 0.50 | 0.50 | 0.50 | 0.50 | 0.50 | 0.50 | 0.50 |
| F | 1.56 | 1.56 | 0.06 | 0.06 | 0.25 | 0.25 | 0.25 | 0.25 | 0.25 | 0.25 | 0.25 | 0.25 |
| G | 0.78 | 0.78 | 0.03 | 0.03 | 0.12 | 0.12 | 0.12 | 0.12 | 0.12 | 0.12 | 0.12 | 0.12 |
| H | SC | SC | SC | SC | SC | SC | SC | SC | SC | SC | SC | SC |

a. “SC” denotes sterility controls (i.e., uninoculated BGM added to test article stocks).
b. “GC” denotes positive growth controls (organism suspension added to wells containing OSB but no test article).
c. Numbers in columns 1-4 denote final lidocaine challenge concentrations in mg/mL; numbers in columns 5-12 denote final challenge concentrations of DMSO or NMP in % (v/v).
d. Note: 100 μL of inoculum (suspended in BGM) was added to 100 μL of diluted 2× test article stocks (excluding row H, which received sterile BGM instead).
e. Lidocaine-HCl (LD-HCL), free-base lidocaine (LD-B), DMSO alone, NMP alone, DMSO (diluted in a constant concentration of 1 mg/mL LD base), or NMP (diluted in a constant concentration of 1 mg/mL LD base) was diluted in columns 1&2, 3&4, 5&6, 7&8, 9&10, and 11&12, respectively (excluding row A).
f. Each plate represented one tested organism; an identical plate layout was used for all seven organisms.

### Calculation and Interpretation of Results

ZOI Diameter:
a. Averaged the two ZOI measurements (i.e., top to bottom and left to right) for each replicate.
b. Averaged the above ZOIs for the three replicates of each test article. Reported the mean±SD of these ZOIs.

Quantitative Results using Log₁₀ Reduction:
a. Counted the appropriate number of colonies on their respective spot plates according to the plating method used. An appropriate number of colonies that was considered viable for counting was a 20 μL spot where the individual colonies were visibly distinct from each other within the plated spot. The section in which this spot was located gave the order of magnitude for the cell enumeration: 10⁰-10⁷.
b. Calculated log₁₀ density of colony forming units (CFU) per mL of inoculum:

Log₁₀ (CFU per mL)=Log₁₀[(X/B)*(D)]
where:
   X=CFU counted per 20-μL spot,
   B=volume plated (0.02 mL), and
   D=dilution
c. Averaged the log₁₀ density for each set of replicates (e.g., 3 LLB, 3 CON, 3 OTS).
d. Calculated the Log₁₀ reduction as follows:
e. Log₁₀ Reduction=Mean Recovery from OTS-Mean Recovery from LLB or CON
f. Log reductions (for test articles relative to control articles) were calculated for the inoculum (i.e., the inoculum in each well, see above step under PLANKTONIC GROWTH/CHALLENGE and LOG REDUCTION ASSAY) to quantitate any inhibitory effects of leaching chemistry against planktonic cells.

MIC determination from Broth Microdilution: MIC results were determined following the overnight incubation of the broth microdilution plates. Ensured that the SC wells were free of growth and that the GC wells were positive for growth. To determine the minimum inhibitory concentration (MIC) values, checked for turbidity in the wells of the challenge plate and scored. The MIC was defined as the minimum concentration that inhibited visible growth of the organism.

### Results

TABLE 5
Inoculum Checks

| Organism | Mean CFU/mL | Log₁₀ CFU/mL ± SD |
|---|---|---|
| P. aeruginosa ATCC 15442 | 1.43 × 10¹⁰ | 10.15 ± 0.01 |
| E. coli ATCC 25922 | 7.25 × 10⁹ | 9.86 ± 0.02 |
| P. mirabilis ATCC 29906 | 5.75 × 10⁹ | 9.76 ± 0.08 |
| S. aureus ATCC 29213 | 4.50 × 10⁹ | 9.63 ± 0.21 |
| S. epidermidis ATCC 35984 | 2.28 × 10⁹ | 9.35 ± 0.06 |
| E. faecalis ATCC 51575 | 5.00 × 10⁹ | 9.66 ± 0.26 |
| C. albicansATCC 10231 | 4.75 × 10⁷ | 7.67 ± 0.10 |

Inoculum Densities (n=2) of O/N Cultures

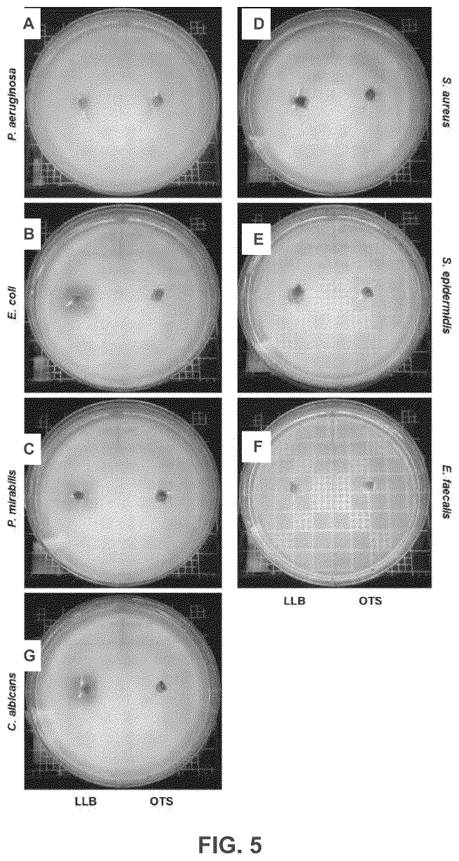

Turning now to FIG. 5 for a series of ZOI assays after 24 hours at 37° C., each comparing a lidocaine loaded band on the left and an off-the-shelf predicate band on the right, for (A) P. aeruginosa ATCC 15442, (B) E. coli ATCC 25922, (C) P. mirabilis ATCC 29906, (D) S. aureus ATCC 29213, (E) S. epidermidis ATCC 25984, (F) E. faecalis ATCC 51575, (G) C. albicans ATCC 10231. “LLB” refers to lidocaine loaded band; and “OTS” refers to off-the-shelf predicate bands.

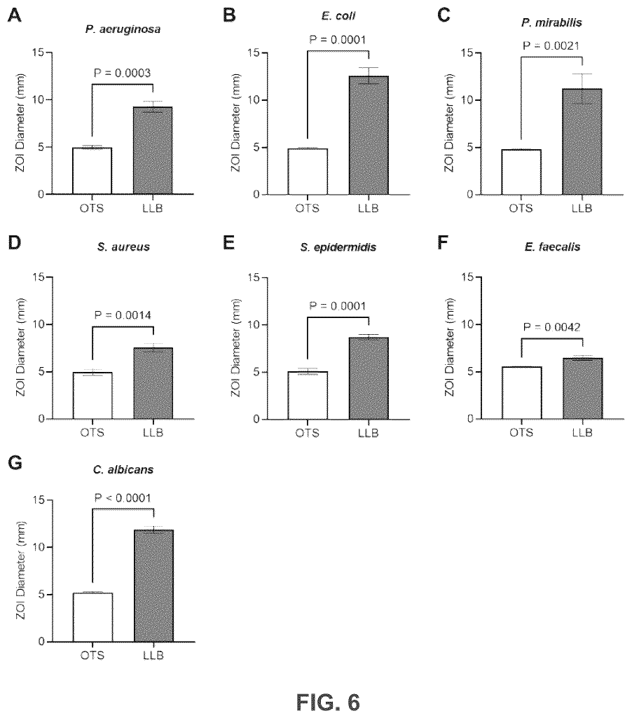

Turning now to FIG. 6 for ZOI quantitation. The outer diameter of each zone of inhibition from FIG. 5 was measured in two directions (left to right and top to bottom) and the average of these was plotted. (A) P. aeruginosa ATCC 15442, (B) E. coli ATCC 25922, (C) P. mirabilis ATCC 29906, (D) S. aureus ATCC 29213, (E) S. epidermidis ATCC 25984, (F) E. faecalis ATCC 51575, (G) C. albicans ATCC 10231. Bars represent the mean±standard deviation for 3 replicate agar plates. P-values were determined with an unpaired, 2-tailed T-test. Note that no ZOI was observed for the off-the-shelf control rings, so the reported ZOI for these rings indicates the cross-section of the article itself. LLB=lidocaine loaded band; OTS=off-the-shelf predicate.

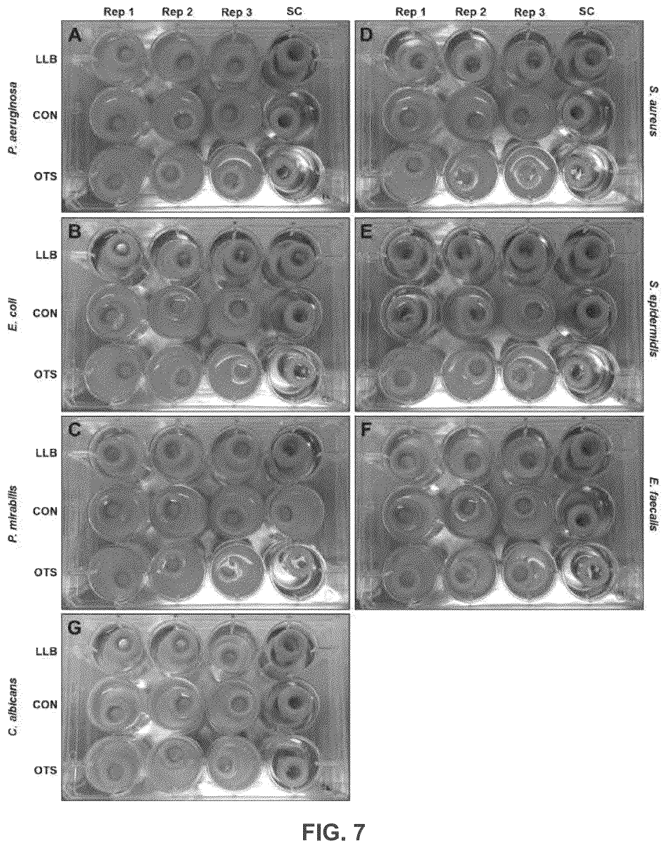

Turning now to FIG. 7 for planktonic inhibition assay. Turbidity of P. aeruginosa (A), E. coli (B), P. mirabilis (C), S. aureus (D), S. epidermidis (E), E. faecalis (F), or C. albicans (G) cultures after exposure to the various elastrator bands for 24 hours. “LLB” refers to lidocaine loaded band; “CON” refers to no lidocaine “mock processed” control; “OTS” refers to off-the-shelf predicate; Rep 1, Rep 2, or Rep 3 denotes the replicate number for each row (i.e., each band type); “SC” refers to sterility control wells (one SC for each band type).

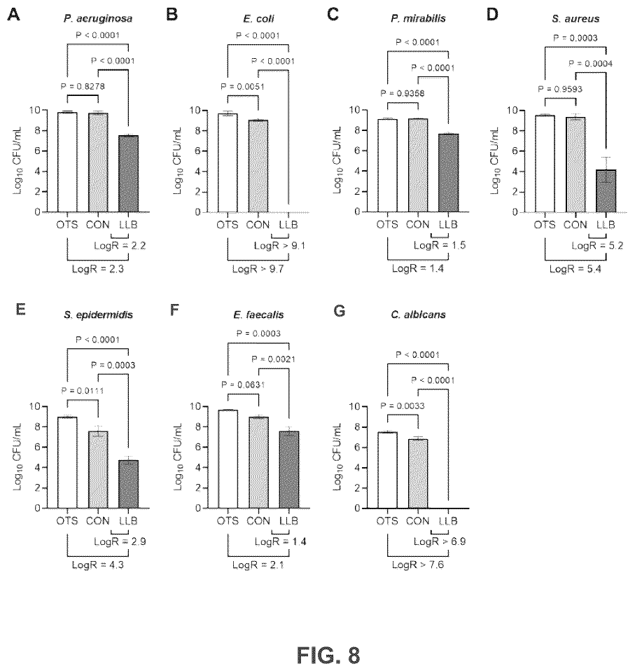

Turning now to FIG. 8 for quantitation of planktonic cell density after exposure to the various elastrator bands for 24 hours. P. aeruginosa ATCC 15442 (A), E. coli ATCC 25922 (B), P. mirabilis ATCC 29906 (C), S. aureus ATCC 29213 (D), S. epidermidis ATCC 25984 (E), E. faecalis ATCC 51575 (F), or C. albicans ATCC 10231 (G) cultures from FIG. 7 were serially diluted, spot plated, and CFU/mL enumerated. “LLB” refers to lidocaine loaded band; “CON” refers to no lidocaine “mock processed” control; “OTS” refers to off-the-shelf predicate; “LogR” refers to log₁₀ reduction. Bars represent the mean±standard deviation for 3 replicate wells. The P-values reported above each graph were determined by one-way ANOVA and Tukey's multiple comparisons test.

TABLE 6
Broth Microdilution Assay (MIC)

| | MIC | | | | | |
|---|---|---|---|---|---|---|
| | LD-HCl | LD-B | DMSO | NMP | DMSO | NMP |
| Organism | (mg/mL) | (mg/mL) | (% v/v) | (% v/v) | +1 mg/mL LD | +1 mg/mL LD |
| P. aeruginosa | 25.0 | >1.00 | >4.00 | 4.00 | >4.00 | 4.00 |
| ATCC 15442 | | | | | | |
| E. coli | 6.25 | >1.00 | >4.00 | 4.00 | >4.00 | 4.00 |
| ATCC 25922 | | | | | | |
| P. mirabilis | 12.5 | >1.00 | >4.00 | 4.00 | >4.00 | 4.00 |
| ATCC 29906 | | | | | | |
| S. aureus | 12.5 | >1.00 | >4.00 | 4.00 | >4.00 | 4.00 |
| ATCC 29213 | | | | | | |
| S. epidermidis | 25.0 | >1.00 | >4.00 | 4.00 | >4.00 | 4.00 |
| ATCC 35984 | | | | | | |
| E. faecalis | 25.0 | >1.00 | >4.00 | 4.00 | >4.00 | 4.00 |
| ATCC 51575 | | | | | | |
| C. albicans | 12.5 | >1.00 | >4.00 | 4.00 | >4.00 | 4.00 |
| ATCC 10231 | | | | | | |

a. MICs for lidocaine-HCl, free-base lidocaine, DMSO, NMP, DMSO+1 mg/mL lidocaine base, or NMP+1 mg/mL lidocaine base against the various organisms.
b. Note: 2× stock concentrations of each test article were prepared in TSB: lidocaine-HCl, 50 mg/mL; lidocaine base, 2 mg/mL (i.e., the practical solubility limit); DMSO, 8% v/v; NMP, 8% v/v; 8% solutions of DMSO or NMP were also prepared in TSB containing 2 mg/mL LD base. The 2× stock solutions were serially diluted (2-fold series) in TSB (or, for the latter two solutions, in TSB+2 mg/mL LD base) before mixing 0.1 mL of each concentration with 0.1 mL of ˜10⁵ CFU/mL organism suspension (in BGM), yielding a final challenge concentration range of 250.78 mg/mL LD-HCl, 1-0.03 mg/mL LD base, 4-0.125% DMSO or NMP, and 4-0.125% DMSO or NMP plus a constant concentration of 1 mg/mL LD base. Sterility controls (i.e., uninoculated BGM added to test articles) were all clear; untreated growth controls (i.e., organisms in BGM added to TSB) were all turbid.
c. Note Also: The broth from FIG. 7 was sent for analysis of the level of IPM and THE by GC-MS, and free-base lidocaine by HPLC, eluted from the bands. IPM and THF were immeasurably low; lidocaine base averaged 2.93 mg/mL, which is substantially lower than the MICs achieved for lidocaine-HCl in this Table and suggesting enhanced antimicrobial activity of LD in the context of the LLBs.

### Conclusions

Regarding inoculum checks, above Table 5 summarizes the inoculum densities. The saturated “overnight” (O/N) cultures reached an average density of approximately 10.15, 9.86, 9.76, 9.63, 9.35, 9.66, and 7.67 log₁₀ CFU/mL for P. aeruginosa, E. coli, P. mirabilis, S. aureus, S. epidermidis, E. faecalis, and C. albicans, respectively.

Regarding ZOI assay, no ZOI was formed by the OTS predicate rings for any of the seven tested organisms, while the LLBs formed striking ZOIs for E. coli, P. mirabilis, S. epidermidis, and C. albicans, and minimal ZOIs for P. aeruginosa, S. aureus, and E. faecalis (FIG. 5). Upon quantitation of the ZOI diameters, LLBs formed significantly larger ZOIs (relative to the OTS predicate bands, which all averaged about 5.1 mm) for all seven tested organisms, although the magnitudes varied from about 6.5 mm to 12.6 mm (FIG. 6). In decreasing order of ZOI diameter: E. coli>C. albicans>P. mirabilis>P. aeruginosa>S. epidermidis>S. aureus>E. faecalis.

Regarding planktonic inhibition assay, the broth was clear in almost every sterility control well; out of a total of 21 SC wells, just one (the “CON” well of the P. mirabilis plate) was turbid (FIG. 7). All wells exposed to OTS controls or mock-processed “no lidocaine” controls were turbid (FIG. 7). A visible reduction in turbidity was observed for the LLB wells for all seven tested organisms, although it was least striking for P. aeruginosa (FIG. 7).

The log₁₀ CFU/mL reductions in the broth from FIG. 7 are presented in FIG. 8. Relative to the OTS predicate controls, the mock processed controls (containing THF and IPM but no Lidocaine) only yielded statistically significant (P<0.05) log reductions greater than 1 for S. epidermidis (FIG. 8D). In contrast, the LLBs yielded statistically significant log reductions (relative to either the mock processed controls or the predicate controls) greater than 1 for all seven tested organisms (FIG. 8). Together, these data indicate that the processed bands lack a significant antimicrobial effect in the absence of lidocaine, although it is possible that LD still requires one of the other components (IPM or THF) for full potency. LLBs yielded log₁₀ reductions, relative to the predicate control bands, of >9.7 (“max kill”), >7.6 (“max kill”), 5.4, 4.3, 2.3, 2.1, and 1.4 for E. coli, C. albicans, S. aureus, S. epidermidis, P. aeruginosa, E. faecalis, and P. mirabilis, respectively.

Notably, each organism had a starting inoculum density of ˜10⁶ CFU/mL, which climbed over the 24-hour incubation period to ˜10⁹ (for the bacteria) or 10⁷ (for the yeast) in the predicate control wells; however, the LLBs reduced this density to ˜10⁴, ˜10⁴, 0, and 0 for S. aureus, S. epidermidis, E. coli, and C. albicans, respectively, indicating that LLBs elute a microbicidal agent for these four organisms. LLBs may only elute an inhibitory agent for P. aeruginosa, P. mirabilis, and E. faecalis.

Regarding broth microdilution (MIC) assay, all sterility controls were clear, and all growth controls were turbid. Because LD-HCl is water soluble, a range of relatively high concentrations could be tested as a positive control. Indeed, the MIC could be observed for all seven tested organisms. The MIC for lidocaine-HCl was 25.0, 6.25, 12.5, 12.5, 25.0, 25.0, and 12.5 mg/mL for P. aeruginosa, E. coli, P. mirabilis, S. aureus, S. epidermidis, E. faecalis, and C. albicans, respectively (Table 6).

This is generally consistent with published literature, which indicates that the MIC for lidocaine-HCl is >20.0, 5.00, and 20.0 mg/mL for P. aeruginosa ATCC 27853, E. coli ATCC 25922, and S. aureus ATCC 29213, respectively.

Unlike lidocaine-HCl, free base lidocaine has limited aqueous solubility, limiting the maximum challenge concentration to 1 mg/mL. All organisms grew at the highest tested concentration of lidocaine base, indicating that the MIC is greater than 1 mg/mL for all seven tested organisms (Table 6).

Although IPM is immiscible with TSB, two alternative permeation enhancers, DMSO and NMP, were each tested at a maximum challenge concentration of 4% v/v. All seven organisms grew at the maximum concentration of DMSO, indicating an MIC of greater than 4% v/v for DMSO (Table 6). In contrast, all seven organisms were inhibited at the highest concentration of NMP, indicating an MIC of 4% v/v for NMP (Table 6).

Finally, to test whether either permeation enhancer displayed synergy with LD base (i.e., had a lower MIC, indicating enhanced potency), the titrations were performed in the presence of a constant concentration of 1 mg/mL lidocaine base. Again, the MIC for DMSO (plus lidocaine base) or NMP (plus lidocaine base) was >4 and =4% v/v, respectively (Table 6), indicating no measurable synergy.

The levels of IPM and THF eluted from the bands into the growth medium (FIG. 7) were all below the minimum reporting levels; the level of free-base lidocaine eluted from the bands into the growth medium (FIG. 7) averaged 2.9 mg/mL, which was substantially lower than the MICs for lidocaine-HCl reported in Table 6. This suggests enhancement of the antimicrobial efficacy of the lidocaine when delivered in association with the bands.

### Concluding Remarks

Relative to the unprocessed predicate bands, lidocaine-loaded bands formed ZOIs of varying sizes for all seven tested organisms. In decreasing order of ZOI diameter: E. coli>C. albicans>P. mirabilis>P. aeruginosa>S. epidermidis>S. aureus>E. faecalis.

Relative to the unprocessed predicate bands, lidocaine-loaded bands significantly inhibited planktonic growth of all seven tested organisms, yielding log₁₀ CFU/mL reductions of >9.7 (“max kill”), >7.6 (“max kill”), 5.4, 4.3, 2.3, 2.1, and 1.4 for E. coli, C. albicans, S. aureus, S. epidermidis, P. aeruginosa, E. faecalis, and P. mirabilis, respectively, after 24 hours of incubation. This effect was almost certainly microbicidal (rather than merely bacteriostatic), at least for E. coli, C. albicans, S. aureus, and S. epidermidis. Moreover, the processed bands did not elute a significant antimicrobial effect in the absence of lidocaine, although it cannot be ruled out that LD requires one of the other components (IPM or THF) for full potency.

The MIC of lidocaine-HCl was 6.25, 12.5, 12.5, 12.5, 25.0, 25.0, and 25.0 mg/mL for E. coli, C. albicans, P. mirabilis, S. aureus, P. aeruginosa, S. epidermidis, and E. faecalis, respectively. The MIC of lidocaine base was >1 mg/mL for all seven tested organisms. The MIC of DMSO and NMP was >4% and =4%, respectively, for all seven tested organisms; these MICs did not decrease in the presence of 1 mg/mL lidocaine base, indicating an absence of measurable synergy.

The levels of free-base lidocaine eluted from the bands into the growth medium during the planktonic inhibition experiment averaged 2.9 mg/mL, which was substantially lower than the MICs for lidocaine-HCl measured in the broth microdilution assay, suggesting some enhancement of lidocaine's antimicrobial efficacy in the context of the bands.

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

It should be understood that the compositions and methods are described in terms of “comprising,” “containing,” or “including” various components or steps, the compositions and methods can also “consist essentially of or “consist of the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are dis-cussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be referenced herein, the definitions that are consistent with this specification should be adopted.

Many obvious variations of the embodiments set out herein will suggest themselves

to those skilled in the art in light of the present disclosure. Such obvious variations are within the full intended scope of the appended claims.

1. A method for enhancing antimicrobial efficacy of a local anesthetic, the method comprising:
(a) providing a drug-eluting medical device containing the local anesthetic, the drug-eluting medical device comprising the local anesthetic within an elastomeric material; and
(b) administering the drug-eluting medical device to a subject such that it contacts a tissue and elutes the local anesthetic thereto;
wherein the eluted local anesthetic provides an enhanced antimicrobial efficacy as compared to the same local anesthetic when not delivered by the drug-eluting medical device.

2. The method of claim 1, wherein the drug-eluting medical device is a tube, a ring, or a band.

3. The method of claim 1, wherein the drug-eluting medical device is a topical, implanted, or indwelling medical device.

4. The method of claim 1, wherein the drug-eluting medical device is a urinary catheter.

5. The method of claim 1, wherein the local anesthetic is a hydrophobic, a hydrophilic, or a lipophilic local anesthetic.

6. The method of claim 1, wherein the local anesthetic is a base form or hydrochloride form of lidocaine, bupivacaine, proparacaine, benzocaine, mepivacaine, bupivacaine liposomal, ropivacaine, chloroprocaine hydrochloride, levobupivacaine, or any combination thereof.

7. The method of claim 6, wherein the local anesthetic is lidocaine.

8. The method of claim 7, wherein the lidocaine is free base lidocaine.

9. The method of claim 6, wherein the local anesthetic is bupivacaine.

10. The method of claim 1, wherein the tissue is epithelial tissue.

11. The method of claim 10, wherein the epithelial tissue is skin.

12. The method of claim 1, wherein the drug-eluting medical device further contains a tissue permeator within the elastomeric material.

13. The method of claim 12, wherein the tissue permeator is isopropyl myristate, fatty acids, fatty acid esters, poloxamers, triglycerides, n-methyl pyrrolidone, terpineol, limonene, dimethyl sulfoxide, dimethylacetamide, or any combinations thereof.

14. The method of claim 1, wherein the elastomeric material is a rubber or polymeric material selected from a natural rubber, a synthetic rubber, a silicone, a polybutadiene, a polyisoprene, a polychloroprene, a nitrile, a poly(styrene-butadiene-styrene) (SBS), a styrene-ethylene-butylene-styrene (SEBS), an ethylene-propylene-diene monomer rubber (EPDM), a polyurethane, a woven material, a natural fiber, a synthetic fiber, and any combination thereof.

15. The method of claim 14, wherein the rubber or polymeric material is latex rubber.

16. The method of claim 1, wherein the drug-eluting medical device further contains a vasoconstrictor within the elastomeric material.

17. The method of claim 16, wherein the vasoconstrictor is epinephrine, pseudoephedrine, phenylephrine, thromboxane, or angiotensin.

18. The method of claim 1, wherein the drug-eluting medical device further contains an antimicrobial agent within the elastomeric material.

19. The method of claim 18, wherein the antimicrobial agent is high valency silver or silver sulphadiazine.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)
